(19) United States
(12) Patent Application Publication
(10) Pub. No.: US 2025/0011667 A1
(43) Pub. Date: Jan. 9, 2025

(54) PRODUCTION OF POLY-ALPHA-OLEFINS

(71) Applicant: Neste Oyj, Espoo (FI)

(72) Inventors: Jan WAHLSTRÖM, Porvoo (FI); Kim VIKMAN, Porvoo (FI)

(73) Assignee: Neste Oyj, Espoo (FI)

(21) Appl. No.: 18/764,493

(22) Filed: Jul. 5, 2024

(30) Foreign Application Priority Data
Jul. 7, 2023 (FI) 20235804

(51) Int. Cl. C10G 69/12; C10G 69/04

(52) U.S. Cl. CPC C10G 69/126; C10G 69/04; C10G 2300/1088; C10G 2300/4006; C10G 2300/4012; C10G 2300/70

(57) ABSTRACT

The present disclosure describes a process for producing poly-α-olefins. The process includes providing at least one α-olefin monomer and a catalyst, and letting the at least one α-olefin monomer react in the presence of the catalyst to form a mixture of poly-α-olefins (PAO). The obtained mixture of poly-α-olefins is fractionated to two PAO fractions wherein the two PAO fractions are a first fraction of PAO having a kinematic viscosity of 5 cSt or lower and a second fraction of PAO having a kinematic viscosity of more than 5 cSt. Optionally a recycle fraction is also obtained in the fractionation and at least a part of the recycle fraction including dimers of the α-olefins can be recycled back to the reaction step.

### FIELD OF THE INVENTION

The present invention relates to a process for producing poly-α-olefins from α-olefin monomers. More particularly the invention relates to a process for producing poly-α-olefins where the product mixture is fractionated to two product fractions and the product fractions are hydrogenated separately.

The present invention therefore provides a simple process, which can be continuous, for producing poly-α-olefins of high quality, with good yield and optimal process and equipment set-up.

### BACKGROUND OF THE INVENTION

Oligomerisation reaction of α-olefins to form various grades of components useful in production of synthetic lubricants are well known. The production method generally includes oligomerisation of the α-olefin monomers using a catalyst or a catalyst complex, removal of the catalyst or catalyst complex from the reaction product and various post-reaction treatments of the oligomerisation product. The oligomerisation product is generally named poly-α-olefin (PAO) and can be categorised based on the viscosity as measured in cSt of the product in the typical range of 2 to 100 cSt. The viscosities of typical low viscosity PAO products are 2, 4, 6 or 8 cSt.

In the oligomerisation of α-olefins dimers, trimers, tetramers, pentamers etc. are formed. Heavier products, i.e. having a higher number of monomer units in the oligomer product, naturally have a higher viscosity compared to lighter products. PAO products are used in various applications and depending on the use, various viscosity and other properties are required of the PAO product. Different oligomers can also be mixed to form products with desired properties.

Publication U.S. Pat. No. 4,434,309 describes oligomerisation of low molecular weight alpha olefins to produce synthetic lubricant base stock. The oligomerisation is performed in the presence of a catalyst, which can be boron trifluoride and a protonic promoter.

One key aspect of the production process is the separation of the catalyst or catalyst complex from the oligomerisation product. Publication EP1694439 describes a typical PAO production process and separation of a catalyst complex containing boron trifluoride (BF3) and alcohol from the oligomerisation product with distillation under reduced pressure.

The produced oligomerisation product typically needs to be hydrogenated in order to saturate any double bonds still present in the poly-α-olefin (PAO). A typical process sequence to hydrogenate an unsaturated product is that the product intermediate is hydrogenated directly after the process reaction section and the hydrogenation is followed by at least one separation step to obtain the desired saturated product fractions. Alternatively, the product is first separated to various product streams with desired properties, which product streams are then subsequently hydrogenated. However, this process sequence requires intermediate storage capacities and campaign wise batch hydrogenation.

There is still a need to develop optimal process sequences in PAO production to enable high quality products with sufficient and economical equipment and process set-ups, with high plant capacity and low downtime.

### BRIEF DESCRIPTION OF THE INVENTION

An object of the present invention is thus to provide a robust process for obtaining poly-α-olefins, with optimal product fractionation and hydrogenation steps. The objects of the invention are achieved by a process characterised by what is stated in the independent claim. The preferred embodiments of the invention are disclosed in the dependent claims.

The invention thus provides a process for producing poly-α-olefins, the process comprising
a) providing at least one α-olefin monomer and a catalyst,
b) letting the at least one α-olefin monomer react in the presence of the catalyst to form a mixture of poly-α-olefins (PAO),
c) fractionating the obtained mixture of poly-α-olefins to two PAO fractions, wherein the two PAO fractions are a first fraction of PAO having a kinematic viscosity of 5 cSt or lower and a second fraction of PAO having a kinematic viscosity of more than 5 cSt; and optionally a recycle fraction, of which at least a part is recycled back to the reaction step, and the recycle fraction comprises dimers of the α-olefins,
d) subjecting the obtained two PAO fractions to separate hydrogenation to obtain hydrogenated PAO fractions, and
e) optionally further fractionating at least one of the hydrogenated PAO fractions to obtain hydrogenated PAO sub-fractions.

An advantage of the current invention is that it provides a process for producing poly-α-olefins with an optimal process and equipment set-up. In one embodiment the presented process provides possibilities to run the oligomerisation and hydrogenation steps continuously with little or no intermediate storage of non-hydrogenated products.

### BRIEF DESCRIPTION OF THE DRAWINGS

In the following the invention will be described in greater detail by means of preferred embodiments with reference to the accompanying figures, in which

FIG. 1 is a schematic process description of one embodiment of the current invention, where the oligomerisation, fractionation and hydrogenation steps are described.

FIG. 2 shows the cracking of various hydrogenated PAO products in different temperatures.

FIG. 3 shows the fractionation temperature required for various PAO products.

### DETAILED DESCRIPTION OF THE INVENTION

It is typically optimal to hydrogenate the total product stream obtained from a process, before separation to final products, especially when the final products are not sensitive to the separation conditions. Poly-α-olefin products (PAO), however, have stringent product specifications, which cannot be met if product degradation is caused in the final process step. For that reason, a typical process sequence for poly-α-olefin production is to provide the hydrogenation step as the last essential process step of poly-α-olefin processing, leading to the challenge that all fractionated intermediated products must be hydrogenated separately. This can be arranged either in separate parallel and continuous units for each product or in one campaign batch wise operating unit.

A beneficial production process concept is based on an optimal equipment arrangement with a minimized required amount of equipment, which continuously performs desired once-through processing efficiently with minimized re-processing of feedstock and intermediate process streams. A continuous process concept will enable continuous full use of equipment at designed and optimally controlled operation conditions, thereby also minimizing unnecessary processing upsets and equipment maintenance.

Batch wise production process concepts have the advance of enabling processing of multiple quality products in the same minimized amounts of equipment. However, the equipment is laborious to operate and is only part-time in full use. In addition, transient modes in batch wise production lower the total yield of products and cause need for reprocessing. Therefore, batch wise production is typically beneficial only in small scale production processes and when several types of products need to be processed in the same batch equipment in campaign modes.

The aim of the current invention is therefore to provide a solution to the described problems related on one hand to a continuous process and on the other hand related to a batch wise process. An object of the present invention is thus to provide a robust process, with optimal product fractionation and hydrogenation steps for obtaining poly-α-olefins. The current invention relates to a process for producing poly-α-olefins (PAO) from α-olefins. The term “α-olefin” is here meant to denote the same as alpha-olefin (or alfa-olefin), and is a 1-alkene, i.e. having a double bond in the primary carbon, also called a carbon. The carbon chain of the α-olefin is preferably linear, i.e. n-alkene. If not specifically mentioned that the α-olefin is branched, it is to be assumed that a linear α-olefin is meant. In one embodiment of the current invention the α-olefin monomer comprises C₄-C₂₀ α-olefins, preferably C₈-C₁₂ α-olefins and more preferably the monomer comprises C₁₀ α-olefin (1-decene). It should be noted that even if the feedstock for the oligomerisation reaction comprises primary e.g. of C₁₀-α-olefin (1-decene) the feedstock is never completely pure 1-decene and always contains various amounts of other olefins. The other olefins will also react in the oligomerisation reaction. Therefore, the poly-α-olefin (PAO) product formed in the oligomerisation reaction is always a mixture comprising products with different degree of oligomerisation (di-, tri, tetramers etc.) and oligomers from other olefin monomers than the primary chosen α-olefin of the feedstock.

The product of the oligomerisation reaction of the α-olefin monomers is called poly-α-olefin (PAO) or poly-alfa-olefin, poly-alpha-olefin, polyalphaolefin or polyalfaolefin. The different oligomerisation products can be characterised based on the numbers of monomer units in the oligomer, i.e. dimers, trimers, tetramers etc. The oligomerisation product can also be characterised based on the viscosity value as expressed in cSt of the product. Typical viscosities of light PAO products include but are not limited to 2, 4, 6, 8 etc kinematic viscosity at 100° C. in cSt.

A PAO product with a certain kinematic viscosity, such as 4 cSt or 6 cSt is always a mixture of oligomers. This is because a product with a certain kinematic viscosity is wanted, and the aim is not to obtain products with only one type of oligomers. It is the properties of the products which are significant, not the chemical structure as such. Products with specific properties, such as kinematic viscosities, are desired.

Therefore, the formed PAO product mixture typically needs to be fractionated to obtain products with the desired properties. The oligomerisation reaction always produces a mixture of oligomers and the final PAO products are formed typically by fractionating the PAO reaction mixture to obtain products with desired properties, such as viscosity and volatility. A fractionation is also typically needed to separate unreacted monomers and/or low oligomerisation products, such as dimers, which should be separated from the product mixture and is typically recycled back to the oligomerisation step. In addition, the catalyst of catalyst complex needs to be removed from the product mixture, this is typically performed before the PAO fractionation.

The oligomerisation reaction of the α-monomers also requires presence of a catalyst. In the current process the catalyst can be any suitable catalyst capable of catalysing the oligomerisation of α-monomers to form poly-ca-oligomers. In one embodiment of the current invention the catalyst is a catalyst complex comprising a primary catalyst and a co-catalyst. The term “catalyst complex” is here used to denote a combination of a catalyst and a co-catalyst (which can also be called a protonic promoter). The catalyst complex can be a physical mixture, or the catalyst and co-catalyst can be bounded with each other, or it can be a combination of both. The primary catalyst can be an aluminium halide or a boron halide, preferably the primary catalyst is boron trifluoride (BF3). The co-catalyst can be C₁-C₁₀ alcohols, or C₁-C₅ alcohols, preferably the co-catalyst is n-butanol.

The performance and advantages of the current invention is not dependent on the chosen α-olefin or catalyst or alternatively catalyst complex used in the oligomerisation. However, in one embodiment of the invention the monomer is linear 1-decene and the catalyst complex is formed from BF3 as primary catalyst with n-butanol as co-catalyst.

The process according to the current invention comprises a step of letting the at least one α-olefin monomer react in an oligomerisation reaction in the presence of the catalyst to form a mixture of poly-α-olefins (PAO) and fractionating the obtained mixture to two PAO fractions and possible a recycle fraction. At least part of the recycle fraction is recycled back to the reaction step for further oligomerisation. The recycle fraction typically comprises at least dimers but possibly also unreacted monomers. The recycle fraction can also be more than one stream and separation of the recycle fraction can comprise more than one separation step. Prior to the fractionating step to obtain PAO fractions, the catalyst or catalyst complex and possible unreacted monomers are removed from the product mixture outlet from the oligomerisation reaction.

The mixture of poly-α-olefins obtained in the oligomerisation reaction is fractionated in a fractionating step to obtain two PAO fractions.

The two PAO fractions are a first fraction of PAO having a kinematic viscosity of 5 cSt or lower and a second fraction of PAO having a kinematic viscosity of more than 5 cSt. The first fraction of PAO having a kinematic viscosity of 5 cSt or lower can also be called a lighter fraction and the second fraction of PAO having a kinematic viscosity of more than 5 cSt can also be called a heavier fraction. In one embodiment more than two PAO fractions are obtained in the fractionation of the mixture of poly-α-olefins, such as three, four or five PAO fractions.

Herein a fraction of PAO (or PAO fraction) having a kinematic viscosity of 5 cSt or lower, means that the fraction contains PAO product with a measured kinematic viscosity at 100° C. of 5 cSt or lower, meaning the kinematic viscosity can be up to 5 cSt, such as 4.5 cSt, 4 cSt or 3.5 cSt etc. In one embodiment the first fraction of PAO product has a kinematic viscosity of from 2 cSt to 5 cSt. Analogously, a fraction of PAO (or PAO fraction) having a kinematic viscosity or more than 5 cSt herein means a PAO product where the measured kinematic viscosity of the PAO product is higher than 5 cSt, such as 5.5 cSt, 6 cSt or 6.5 cSt etc. In one embodiment the second fraction of PAO product has a kinematic viscosity of from more than 5 cSt to 10 cSt, or more than 5 cSt to 12 cSt. A fraction of PAO product separated as a fraction has one measured kinematic viscosity and the range of the kinematic viscosities of the first and second fractions means that the measured kinematic viscosity of the fraction is within this range.

Alternatively, in one embodiment the first PAO fraction has a kinematic viscosity of 4 cSt or lower and a second PAO fraction has a kinematic viscosity of more than 4 cSt. Still alternatively, in one embodiment the first PAO fraction has a kinematic viscosity of 6 cSt or lower and a second PAO fraction has a kinematic viscosity of more than 6 cSt.

Typically, three different PAO products with different kinematic viscosities are produced in the current process for producing poly-α-olefins according to the invention. These three PAO products are in one embodiment of the invention, a 2 cSt product, a 4 cSt product and either a 6 cSt product or an 8 cSt product. Thereby, the first fraction of PAO product (lighter fraction) comprises two PAO products, namely a 2 cSt and a 4 cSt product, while the second fraction of PAO product (heavier fraction) comprises one PAO product, namely either a 6 cSt product or an 8 cSt product. Thereby the first PAO fraction having a kinematic viscosity of 5 cSt or lower, typically comprises two PAO products, a 2 cSt and a 4 cSt product. Similarly, the second PAO fraction having a kinematic viscosity of more than 5 cSt, typically comprises one PAO product, a 6 cSt or an 8 cSt product.

The current process comprises fractionating the obtained mixture of poly-α-olefins to two PAO fractions and optionally a recycle fraction, of which a part can be recycled back to the reaction step, and the recycle fraction comprises dimers of the α-olefins. It is beneficial to perform the fractionation such that two fractions of approximately equal volumes are obtained. This means that if separate hydrogenation units are used for the two PAO fractions the hydrogenation units can be of equal size and the streams and lining to the hydrogenation units are interchangeable. Two fractions of approximately equal size can be obtained by fractionating the PAO product such that one fraction contains a PAO product having a kinematic viscosity of 5 cSt or lower and a second fraction of PAO having a kinematic viscosity of more than 5 cSt.

It is also beneficial to obtain two fractions, since only the lighter fraction, i.e. having a kinematic viscosity of 5 cSt or lower, will need to be further separated after hydrogenation. This further separation can be performed in mild processing conditions, and will thus not cause product degradation. On the other hand, the heavier fraction, i.e. the fraction having a kinematic viscosity of more than 5 cSt, will be a fully ready product after hydrogenation and no further product degradation can happen. Separation of heavy products needs more stringent operation conditions, which would result in high risk of product degradation.

It was surprisingly found that a fractionation to two PAO products, a first PAO fraction with kinematic viscosity of 5 cSt or lower and a second fraction of PAO having a kinematic viscosity of more than 5 cSt, is beneficial. This is because the further fractionation of the lighter PAO fraction to two separate PAO products, typically a 2 cSt and a 4 cSt product, can be done in lower temperature without product degradation (see examples hereby provided). The heavier PAO fraction again is typically a ready product after hydrogenation, and no further fractionation of the hydrogenated heavier PAO product is needed.

When a heavier PAO product, such as an 8 cSt product, is desired, the total product volume decreases and the operation time is pro-longed. However, it is still beneficial to obtain two PAO fractions, since the desired heavy fraction can be hydrogenated in one hydrogenation unit, while the other lighter fraction, which is significantly lower in volume, when a heavier product such as an 8 cSt product is desired, can be stored in intermediate storage tanks, and the other hydrogenation unit can undergo service and maintenance work. Therefore, obtaining two PAO fractions and hydrogenating the PAO fractions separately in batch-wise manner or having two separate hydrogenation units for the two PAO fractions enables an end-to-end process with optimal product fractionation and hydrogenation steps for obtaining poly-α-olefins.

In one embodiment of the invention, obtaining two PAO fractions enables two hydrogenation processing units with separate lines, which can be interchanged and therefore enables catalyst replacement in one hydrogenation unit during continuous end-to-end processing mode for the other hydrogenation unit.

The kinematic viscosity of a mixture or fraction of PAO product is a typical way of characterising PAO products and mixtures of various poly-α-olefins. A person skilled in the art is well familiar with measuring kinematic viscosity of products such as PAO products. Kinematic viscosities are measured according to ASTM D-445 (latest version) and typically kinematic viscosities are measured at least at temperatures of 100° C., 40° C. and 0° C. Kinematic viscosities herein used are measured at 100° C. if not otherwise defined. The kinematic viscosities are given in cSt (centi Stokes), which corresponds to the SI unit mm²/s.

A PAO fraction having a kinematic viscosity of e.g. 5 cSt or lower herein is denoted to mean that the kinematic viscosity of the fraction has a value at 100° C. of 5 cSt or lower. The PAO fractions are typically mixtures of oligomers and therefore it is useful to define a fraction based on the kinematic viscosity of the fraction, instead of e.g. using number of monomer units in the oligomers. A person skilled in the art is well familiar with obtaining PAO fractions having a specific kinematic viscosity and can therefore perform a fractionation of a mixture of PAO products to obtain a PAO fraction with a specific kinematic viscosity. The fractionation can be performed with any well-known distillation or fractionation procedure, such as vacuum distillation.

The fractionation to two PAO fractions can be performed as a distillation in reduced pressure such that the required temperature is for example at most 280° C., at most 300° C. or at most 330° C. to separate the first PAO fraction with kinematic viscosity of 5 cSt or lower and the second fraction of PAO having a kinematic viscosity of more than 5 cSt. The distillation temperature depends on the kinematic viscosity of the bottom fraction and the reduced pressure. The reduced pressure can be from 0.2 kPa to 1 kPa at the top of the distillation column.

The process according to the invention further comprises a step of hydrogenating the obtained two PAO fractions to obtain hydrogenated PAO fractions. The step of hydrogenation of the two PAO fractions occurs directly after the fractionation and the process thereby comprises a step of direct hydrogenating the two obtained PAO fractions to separate hydrogenation to obtain hydrogenated PAO fractions. Direct hydrogenation of the two PAO fractions means that the PAO fractions are not subjected to further oligomerisation, recycling back to the oligomerisation or other chemical modifications of the fractions before being subjected to the hydrogenation step. Hydrogenation of the PAO fractions are performed separately and can be performed in separate hydrogenation units. Thereby, in one embodiment there are at least two separate hydrogenation units, which work in parallel.

Hydrogenation of PAO products are well known for the person skilled in that art, and the current process is not restricted to any specific hydrogenation process. Typically, the hydrogenation is performed in the presence of a noble metal or nickel catalyst, preferably a nickel catalyst. The hydrogenation can be performed in a fixed bed reactor. In one embodiment the hydrogenation is performed under a hydrogen pressure of from 2 MPa to 10 MPa, preferably from 2.5 MPa to 8 MPa, more preferably from 3 MPa to 6 MPa; a temperature from 100° C. to 320° C., preferably from 120° C. to 280° C., more preferably from 130° C. to 250° C. Typically hydrogen is used at an amount of 25 to 500 Nm³ hydrogen/m³ hydrocarbons, and the LHSV is from 0.2 to 10 1/h.

In one embodiment of the invention the process further comprises a further fractionation step after the hydrogenation of PAO fractions, where sub-fractions of the hydrogenated PAO fractions are obtained. In one embodiment the hydrogenated PAO sub-fractions are obtained from the hydrogenated PAO fraction having a kinematic viscosity of 5 cSt or lower, and the further fractionation to sub-fractions is performed such that a first hydrogenated PAO sub-fraction having kinematic viscosity of about 2 cSt and a second hydrogenated PAO sub-fraction having a kinematic viscosity of about 4 cSt are obtained.

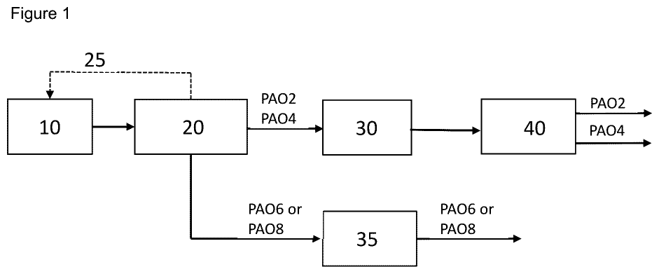

FIG. 1 shows a schematic process of one embodiment of the current invention. The oligomerisation reaction of the α-olefin monomers and catalytic separation is performed in the oligomerisation unit (10) to produce PAO products. The formed PAO products are subjected to a fractionation (20), where two PAO product fractions are obtained. Prior to the fractionation step, the catalyst complex is removed from the product flow (not shown in figure). A first fraction containing PAO products with kinematic viscosities of 2 cSt and 4 cSt and a second fraction containing PAO product with kinematic viscosity of 6 cSt or 8 cSt are obtained. The two PAO fractions are subjected to hydrogenation in separate hydrogenation units, a first hydrogenation unit (30) for the 2 cSt and 4 cSt PAO product fraction and a second hydrogenation unit (35) for 6 cSt or 8 cSt PAO product. From the fractionation unit (20) can also an optional recycle stream (25) of dimers be withdrawn and at least partly recycled back to oligomerisation reaction unit (10). After the first hydrogenation unit (30) the hydrogenated PAO fraction of 2 cSt and 4 cSt PAO product is subjected to a second fractionation unit (40) to separate the 2 cSt and 4 cSt PAO products.

It has surprisingly been found that it is beneficial to perform a fractionation to two PAO product fractions prior to hydrogenation, and that the hydrogenation of the at least two PAO fractions is performed separately or in separate hydrogenation units. This is because a first fractionation to separate a recycle fraction, mainly comprising dimers of the α-olefins, typically needs to be separated from the PAO product mixture before hydrogenation. Also, the fractionation of PAO products will typically result in cracking and possible formation of double bonds or similar and a hydrogenation step is needed after a fractionation step. It is especially advantageous to fractionate the PAO product mixture to two fractions exactly at a kinematic viscosity of 5 cSt and directly hydrogenate these fractions separately. Fractionation at this specific kinematic viscosity and directly hydrogenating the obtained two PAO fractions separately is beneficial because it ensures that each fraction undergoes hydrogenation under conditions that are best suited for their respective properties. Specifically, the PAO fraction with a kinematic viscosity of 5 cSt or lower can undergo a further fractionation step to obtain two PAO sub-fractions with kinematic viscosities of 2 cSt and 4 cSt, respectively. This further separation can be performed under mild processing conditions, thus preventing product degradation, as shown in the Examples below.

Fractionation to more than two fractions could be beneficial, but typically the costs of having more than two hydrogenation units outweigh the benefit of obtaining more than two PAO fractions. If more than two PAO fractions are obtained before hydrogenation, the process should include more intermediate storing of the PAO fractions and the hydrogenation is performed in a semi-batch operation mode.

### Example 1

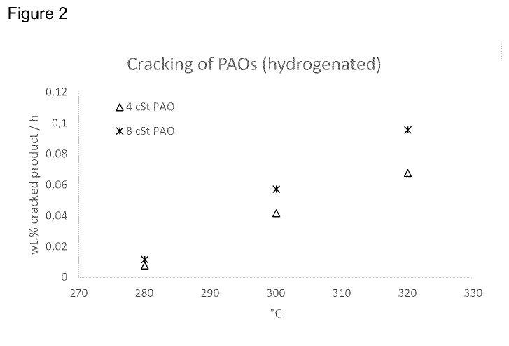

Two PAO products with different kinematic viscosities, namely a 4 cSt PAO and 8 cSt PAO grade, were tested for their thermal stability. The PAO products were charged to similar autoclaves and placed into an oven, which was kept at constant temperature. The autoclaves were left in the oven for several hours. The amount of product left in the autoclave after the test was recorded. The liquid sample was analysed by GC for quantification of cracked products. The tests were repeated for two sets at temperatures 280° C., 300° C. and 320° C. All tests had some colour change. The measured cracking rate is presented in FIG. 2.

The results clearly show that lowering temperature reduces cracking rate almost linearly. It can also be observed that the heavier 8 cSt PAO product has a higher tendency to crack. In other words, the higher the oligomer chain, the more susceptible it is for thermal degradation.

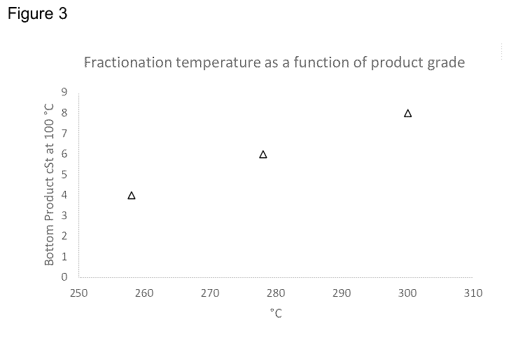

Typical maximum processing temperatures required for fractionation of the various PAO products are presented as a function of product grade in FIG. 3. It can be concluded that fractionating the heavy PAO grades, e.g. 6 cSt and 8 cSt, has a higher risk of product degradation during processing due to the higher temperature required for fractionation.

It will be obvious to a person skilled in the art that, as the technology advances, the inventive concept can be implemented in various ways. The invention and its embodiments are not limited to the examples described above but may vary within the scope of the claims.

1. A process for producing poly-α-olefins, the process comprising:
a) providing at least one α-olefin monomer and a catalyst;
b) reacting the at least one α-olefin monomer in a presence of the catalyst to form a mixture of poly-α-olefins (PAO);
c) fractionating the mixture of poly-α-olefins to two PAO fractions, wherein the two PAO fractions are a first fraction of PAO having a kinematic viscosity of 5 cSt or lower and a second fraction of PAO having a kinematic viscosity of more than 5 cSt; and optionally a recycle fraction, of which at least a part is recycled back to the reacting step, and the recycle fraction includes dimers of the α-olefins;
d) subjecting the two PAO fractions to separate hydrogenations to obtain hydrogenated PAO fractions; and
e) optionally further fractionating at least one of the hydrogenated PAO fractions to obtain hydrogenated PAO sub-fractions.

2. The process according to claim 1, wherein the two PAO fractions are hydrogenated in separate hydrogenation units.

3. The process according to claim 1, comprising:
a further fractionation step e), and the further fractionation step e) is performed on the hydrogenated PAO fraction having a kinematic viscosity of 5 cSt or lower to obtain a first hydrogenated PAO sub-fraction having a kinematic viscosity of about 2 cSt and a second hydrogenated PAO sub-fraction having a kinematic viscosity of about 4 cSt.

4. The process according to claim 1, wherein:
the at least one α-olefin monomer includes C₄-C₂₀ α-olefins, and/or C₈-C₁₂ α-olefins and, and/or C₁₀ α-olefin.

5. The process according to claim 1, wherein;
the catalyst is a catalyst complex includes a primary catalyst and a co-catalyst, and the primary catalyst is an aluminium halide or a boron halide, and/or the primary catalyst is BF3; and
wherein the co-catalyst selected from C₁-C₁₀ alcohols, and/or the co-catalyst is n-butanol.

6. The process according to claim 1, comprising:
performing the hydrogenation in a presence of a noble metal or nickel catalyst in a fixed bed reactor.

7. The process according to claim 1, comprising:
performing the hydrogenation in a pressure from 2 MPa to 10 MPa, and/or from 2.5 MPa to 8 MPa, and/or from 3 MPa to 6 MPa; and
at a temperature from 100° C. to 320° C., and/or from 120° C. to 280° C., and/or from 130° C. to 250° C.
